(19) United States
(12) Patent Application Publication
(10) Pub. No.: US 2017/0095545 A1
(43) Pub. Date: Apr. 6, 2017

(54) INHIBITION OF TUMOR ANGIOGENESIS BY CHECKPOINT INHIBITORS AND ACTIVE VACCINATION

(71) Applicant: Batu Biologics, Inc., San Diego, CA (US)

(72) Inventors: Samuel C. Wagner, San Diego, CA (US); Thomas E. Ichim, San Diego, CA (US); Vladimir Bogin, San Diego, CA (US)

(73) Assignee: Batu Biologics, Inc., San Diego, CA (US)

(21) Appl. No.: 15/149,130

(22) Filed: May 7, 2016

Related U.S. Application Data
(60) Provisional application No. 62/160,106, filed on May 12, 2015.

(51) Int. Cl. A61K 39/00; A61K 45/06

(52) U.S. Cl. CPC A61K 39/0011; A61K 45/06; A61K 2039/515

(57) ABSTRACT

Disclosed are compositions of matter, methods, and protocols useful for treatment of cancer through induction of anti-angiogenic immune responses by vaccination together with immune modulation triggered by checkpoint inhibitors. The invention provides placenta, placental endothelial, placental fibroblasts, and mixtures thereof as immunogens, whose anti-angiogenic activity is augmented by utilization of checkpoint inhibitors. Means of differentiating tumor cells directly into endothelial or endothelial-like cells and utilizing said cells as immunogens for the purpose of inducing immunity against blood vessels feeding tumors. In one embodiment CTLA4 blockade is performed in combination with an immunogen capable of triggering immunity towards tumor endothelial cells. In another embodiment blockade of the PD1-PD1 ligand pathway is performed in combination with induction of anti-angiogenic immune response.

### CROSS-REFERENCE TO RELATED APPLICATION

This application claims the benefit of U.S. Provisional Application No. 62/160,106 filed on May 12, 2015, the contents of which are incorporated herein by reference in its entirety.

### FIELD OF THE INVENTION

The invention pertains to the field of immunotherapy of cancer, more particularly, the invention relates to the field of stimulating immunity to blood vessels associated with the cancer, more specifically, the invention relates to the field of augmenting immunotherapy to cancer by augmentation of a stimulated immune response to tumor associated blood vessels by inhibition of inhibitory signals of the immune response.

### BACKGROUND OF THE INVENTION

The immune system is comprised of multiple different cell types, biologically active compounds and molecules and organs. These include lymphocytes, monocytes and polymorphonuclear leukocytes, numerous soluble chemical mediators (cytokines and growth factors), the thymus, postnatal bone marrow, lymph nodes, liver and spleen. All of these components work together through a complex communication system to fight against microbial invaders such as bacteria, viruses, fungi and parasites, and tumor cells. Together, these components recognize specific molecular antigens as foreign or otherwise threatening, and initiate an immune response against cells or viruses that contain the foreign antigen. The immune system also functions to eliminate damaged or cancerous cells through active surveillance using the same mechanisms used to recognize microbial or viral invaders. The immune system recognizes the damaged or cancerous cells via antigens that are not strictly foreign, but are aberrantly expressed or mutated in the targeted cells.

Unfortunately, while immunity to cancer cells has been demonstrated, this is not effective at a level sufficient to induce clinical responses in many cases. One method of augmenting immune response is to depress the self-regulatory mechanisms that the immune responses uses to regulate itself. Inhibition of inhibitory signals, called “checkpoint inhibitors” have demonstrated promising clinical efficacy in numerous situations.

A clinical study reported by Herbst et al. was designed to evaluate the single-agent safety, activity and associated biomarkers of PD-L1 inhibition using the MPDL3280A, a humanized monoclonal anti-PD-L1 antibody administered by intravenous infusion every 3 weeks (q3w) to patients with locally advanced or metastatic solid tumors or leukemias. Across multiple cancer types, responses as per RECIST v1.1 were observed in patients with tumors expressing relatively high levels of PD-L1, particularly when PD-L1 was expressed by tumor-infiltrating immune cells. Specimens were scored as immunohistochemistry 0, 1, 2, or 3 if <1%, ≧1% but <5%, ≧5% but <10%, or ≧10% of cells per area were PD-L1 positive, respectively. In the 175 efficacy-evaluable patients, confirmed objective responses were observed in 32 of 175 (18%), 11 of 53 (21%), 11 of 43 (26%), 7 of 56 (13%) and 3 of 23 (13%) of patients with all tumor types, non-small cell lung cancer (NSCLC), melanoma, renal cell carcinoma and other tumors (including colorectal cancer, gastric cancer, and head and neck squamous cell carcinoma). Interestingly, a striking correlation of response to MPDL3280A treatment and tumor-infiltrating immune cell PD-L1 expression was observed. In summary, 83% of NSCLC patients with a tumor-infiltrating immune cell IHC score of 3 responded to treatment, whereas 43% of those with IHC 2 only achieved disease stabilization. In contrast, most progressing patients showed a lack of PD-L1 upregulation by either tumor cells or tumor-infiltrating immune cells.

In another study examining the MPDL3280A antibody, Powles et al., treated patients with metastatic urothelial bladder cancer. Responses were often rapid and many occurring at the time of the first response assessment (6 weeks). This study also confirmed that tumors expressing PD-L1-positive tumor-infiltrating immune cells had particularly high response rates. A response rate of 43% (95% CI: 26-63%) achieved in advanced UBC patients with PD-L1 IHC 2/3 tumors provides evidence of noteworthy clinical activity of MPDL3280A. Patients with PD-L1 IHC 0/1 tumors had a response rate of only 11% (95% CI: 4-26%).

Clinical inhibition of CTLA4 has been performed with ipilimumab and tremelimumab. Although these anti-CTLA-4 antibodies have modest response rates in the range of 10%, ipilimumab significantly improves OS, with a subset of patients experiencing long-term survival benefit. In a phase III trial, tremelimumab was not associated with an improvement in OS, and tremelimumab is not currently approved for the treatment of melanoma. Across clinical trials, survival for ipilimumab-treated patients begins to separate from those patients treated in control arms at around 4-6 months, and improved survival rates are seen at 1, 2, and 3 years. Further, in aggregating data for patients treated with ipilimumab, it appears that there may be a plateau in survival at approximately 3 years. Thereafter, patients who remain alive at 3 years may experience a persistent long-term survival benefit, including some patients who have been followed for up to 10 years.

Though cancer immunotherapy approaches have been pursued for decades and have been successful in some cases (e.g. IL-2 in melanoma), checkpoint inhibition and, in particular, PD-1/PD-L1 blockade, is the first strategy that is poised to impact the outcome in cancer patients on a broader scale.

Under physiologic conditions, both stimulatory and inhibitory pathways regulate the inflammatory immune response to pathogens and maintain tolerance to self-antigens. These are regulated by a diverse set of immune checkpoints, thereby protecting healthy tissues from damage. These checkpoints can be co-opted by malignant tumors to dampen the immune response and evade destruction by the immune system. The CTLA-4 and PD-1pathways have been the initial focus of anticancer agent development agents targeting other pathways are also in development.

Generally, the CTLA-4 and PD-1 pathways operate at different stages of the immune response. CTLA-4 modulates the immune response early—at the time of T-cell activation by antigen presenting cells (APCs). T cells are activated by antigen presented on APC in the context of major histocompatibility complex (MHC) (signal 1), as well as co-stimulatory binding of CD28 to B7 (CD80/86) (signal 2). Upon T-cell activation, CTLA-4 is trafficked from the Golgi apparatus to the plasma membrane where it out-competes CD28 in binding to B7 ligands on APCs due to its much higher binding affinity. CTLA-4 binding to B7 ligands inhibits further T-cell activation, limiting the time for T-cell activity. In this way, the magnitude and duration of initial immune responses is physiologically controlled.

In the setting of cancer, inhibiting CTLA-4 may lead to continued activation of a larger number of T cells, resulting in more effective antitumor responses. Preclinical evidence suggests that anti-CTLA-4 antibody can also deplete or inhibit regulatory T cells present in the tumor. CTLA-4 blockade has the potential to activate T cells that are specific for a wide range of antigens, including self-antigens, or deplete regulatory T cells that control autoreactive effector T cells, which may explain the autoimmune-like toxicities observed with CTLA-4 blockade.

In contrast to the effect of CTLA-4 on early T-cell activation, the PD-1 pathway appears to impact the T-cell response at the (later) effector stage. PD-1 is upregulated on T cells after persistent antigen exposure, typically in response to chronic infections or tumors. PD-L1 and PD-L2, the ligands for PD-1, can be expressed by tumor cells, as well as several other hematopoietic and non-hematopoietic cell types. Expression of PD-L1 and PD-L2 is induced by inflammatory cytokines, predominately interferon-γ. In tumors, oncogenic signaling pathways can also upregulate PD-L1 expression. When PD-1 binds its ligand, the T cell receives an inhibitory signal. Over time, chronic inhibition via PD-1:PD-L1 in tumor leads to T-cell anergy and blockade of a productive antitumor immune response.

### BRIEF DESCRIPTION OF THE DRAWINGS

FIG. 1 illustrates anti CTLA-4 antibody given every second day.

FIG. 2 illustrates anti PD-1 antibody given every second day.

### DETAILED DESCRIPTION OF THE INVENTION

The current invention provides novel synergies between agents that immunologically derepress a cancer patient and the tumor vaccines targeting the tumor associated endothelium. One such tumor vaccine is “ValloVax”. ValloVax was previously described by us, and incorporated by reference (Ichim et al., Journal of Translational Medicine 2015) as a placental endothelial derived vaccine capable of inducing immunity to tumor endothelium and thus being effective in animal tumor models irrespective of tissue of origin.

Derepression of tumor immunity involves suppressing a suppressor of the immune response that is produced either by tumor cells, or by host cells responding to tumor cells. Numerous means of tumor immune evasion are known in the art. Effectors of tumor immune evasion include indolamine 2,3 deoxygenase (IDO), IL-4, IL-10, IL-13. Additionally, suppression of the immune response by tumors occurs at the level of the T cell by CTLA4 inhibiting T cell activation, and subsequently the PD1-PD1-ligand interaction inhibiting T cell multiplication and effector function after the T cell has been chronically activated. One embodiment of the invention teaches the combination of inhibitors of checkpoints, such as inhibitors of IDO, IL-4, IL-10, etc together with vaccines that target tumor associated blood vessels. Inhibitors may be classical inhibitors such as antibodies, molecules that induce RNA interference, decoy peptides and small molecules, or non-classical/indirect inhibitors such as agents that suppress GSK3 which in turn render immune cells resistant to the effects of IDO.

Accordingly, in one embodiment of the invention derepression of the immune system is accomplished by administration of epigenetic modifying agents such as valproic acid, 5 azacytine, or trichostatin A in combination with a vaccine targeting tumor endothelial cells. A review of utilization of epigenetic acting agents is given in the following paper and incorporated by reference.

Below are definitions useful for the practice of the invention:

“Antigen-presenting cells” or “APCs” are used to refer to autologous cells that express MHC Class I and/or Class II molecules that present antigens to T cells. Examples of antigen-presenting cells include, e.g., professional or non-professional antigen processing and presenting cells. Examples of professional APCs include, e.g., B cells, whole spleen cells, monocytes, macrophages, dendritic cells, fibroblasts or non-fractionated peripheral blood mononuclear cells (PMBC). Examples of hematopoietic APCs include dendritic cells, B cells and macrophages. Of course, it is understood that one of skill in the art will recognize that other antigen-presenting cells may be useful in the invention and that the invention is not limited to the exemplary cell types described herein. APCs may be “loaded” with an antigen that is pulsed, or loaded, with antigenic peptide or recombinant peptide derived from one or more antigens. In one embodiment, a peptide is the antigen and is generally antigenic fragment capable of inducing an immune response that is characterized by the activation of helper T cells, cytolytic T lymphocytes (cytolytic T cells or CTLs) that are directed against a malignancy or infection by a mammal. In one, embodiment the peptide includes one or more peptide fragments of an antigen that are presented by class I MHC or class II MHC molecules. The skilled artisan will recognize that peptides or protein fragments that are one or more fragments of other antigens may used with the present invention and that the invention is not limited to the exemplary peptides, tumor cells, cell clones, cell lines, cell supernatants, cell membranes, and/or antigens that are described herein.

“Dendritic cell” or “DC” refer to all DCs useful in the present invention, that is, DC is various stages of differentiation, maturation and/or activation. In one embodiment of the present invention, the dendritic cells and responding T cells are derived from healthy volunteers. In another embodiment, the dendritic cells and T cells are derived from patients with cancer or other forms of tumor disease. In yet another embodiment, dendritic cells are used for either autologous or allogeneic application.

“Effective amount” refers to a quantity of an antigen or epitope that is sufficient to induce or amplify an immune response against a tumor antigen, e.g., a tumor cell.

“Vaccine” refers to compositions that affect the course of the disease by causing an effect on cells of the adaptive immune response, namely, B cells and/or T cells. The effect of vaccines can include, for example, induction of cell mediated immunity or alteration of the response of the T cell to its antigen.

“Immunologically effective” refers to an amount of antigen and antigen presenting cells loaded with one or more heat-shocked and/or killed tumor cells that elicit a change in the immune response to prevent or treat a cancer. The amount of antigen-loaded and/or antigen-loaded APCs inserted or reinserted into the patient will vary between individuals depending on many factors. For example, different doses may be required for an effective immune response in a human with a solid tumor or a metastatic tumor.

The terms “nucleic acid” and “oligonucleotide” are used interchangeably herein to mean multiple nucleotides (i.e., molecules comprising a sugar (e.g., ribose or deoxyribose) linked to a phosphate group and to an exchangeable organic base, which is either a substituted pyrimidine (e.g., cytosine (C), thymidine (T) or uracil (U)) or a substituted purine (e.g., adenine (A) or guanine (G)). As used herein, the terms refer to oligodeoxyribonucleotides, oligoribonucleotides (i.e., a polynucleotide minus the phosphate) and any other organic base containing polymer. Nucleic acid molecules can be obtained from existing nucleic acid sources (e.g., genomic or cDNA), but are preferably synthetic (e.g., produced by nucleic acid synthesis).

As used herein, the term “treat”, “treated” or “treating” when used with respect to an infectious disease refers to a prophylactic treatment which increases the resistance of a subject (a subject at risk of infection) to infection with a pathogen, or in other words, decreases the likelihood that the subject will become infected with the pathogen as well as a treatment after the subject (a subject who has been infected) has become infected in order to fight the infection, e.g., reduce or eliminate the infection or prevent it from becoming worse.

The treatment of a subject or with an immunostimulatory oligonucleotide together with checkpoint inhibition or as a means of stimulating checkpoint inhibition is described herein, results in the reduction of infection or the complete abolition of the infection, reduction of the signs/symptoms associated with a disorder associated with a self antigen or the complete abolition on the disorder, or reduction of the signs/symptoms associated with a disorder associated with an addictive substance or the complete abolition of the disorder.

An “antigen” as used herein is a molecule that is capable of provoking an immune response. Antigens include, but are not limited to, cells, cell extracts, proteins, recombinant proteins, purified proteins, polypeptides, peptides, polysaccharides, polysaccharide conjugates, peptide and non-peptide mimics of polysaccharides and other molecules encoded by plasmid DNA, haptens, small molecules, lipids, glycolipids, carbohydrates, whole killed pathogens, viruses and viral extracts, live attenuated virus or viral vector, live attenuated bacteria or a bacterial vector and multicellular organisms such as parasites and allergens. The term antigen broadly includes any type of molecule which is recognized by a host immune system as being foreign or damaged/mutated/overexpressed self proteins.

In some embodiments, the present invention provides a method of determining therapeutic response in cancer patients undergoing ValloVax treatment, the method comprising the steps of: i. obtaining a baseline level of antibody reactive to one or more predetermined biomarker antigens (e.g., non-target predetermined biomarker antigens), said predetermined biomarker antigens, for example, selected from the group consisting of KLK2, KRAS, ERAS, LGALS8, LGALS3, and PSA; administering to the cancer patient T cells activated ex vivo using a protein comprising ValloVax; iii. obtaining a post-treatment antibody level reactive to the one or more predetermined biomarker antigens (e.g., non-target predetermined biomarker antigens) from a patient blood sample after treating with the activated T cells; and, iv. measuring differences between the baseline and post-treatment reactive antibody levels to the one or more predetermined biomarker antigens (e.g., non-target predetermined biomarker antigens) where an increase in antibody level for the predetermined biomarker antigens (e.g., non-target predetermined biomarker antigens) over their baseline level predicts a positive therapeutic response.

In some embodiments, the present invention provides a method of identifying target endothelial antigens for cancer treatment with improved patient survival, the method comprising: i. obtaining baseline level of antibody reactive to one or more biomarker antigens; ii. treating a patient suffering from cancer with ValloVax immunotherapy; iii. obtaining a post-treatment level of antibody reactive to the one or more biomarker antigens from a patient blood sample after treating with the cancer immunotherapy; iv. comparing the baseline and post-treatment reactive antibody levels to determine one or more biomarker antigens in which the reactive antibody level is increased; v. correlating the increase in the one or more biomarker antigen reactive antibody levels to an increase in survival; and vi. identifying the one or more biomarker antigens in which the increase in reactive antibody levels are correlated with survival as target antigens for cancer treatment with improved patient survival.

It is suggested that tumor cell death or tissue damage during the initial response to a cancer immunotherapy may lead to the release and priming of self-reactive T and/or B lymphocytes specific to antigens that are not directly targeted by the therapy. The broadened immune response may subsequently promote more efficient killing of tumor cells (Hardwick, et al., 2011; Corbiere, et al., 2011), including those that may not express the tumor antigen targeted by the immunotherapy (Santegoets, 2011). Early studies in this area have suggested that a broadened antibody response to a cancer immunotherapy may be observed at a higher frequency in clinical responders compared to non-responders (Santegoets, 2011; Butterfield, et al., 2003; T. C. Harding M N, et al., 2008; Mittendorf, et al., 2006). Antigen spread (i.e., antibody responses to antigen/s that are not contained in the immunotherapy) has been observed in response to target-specific cancer vaccines and immunotherapy treatments such as PSA immunotherapy for prostate cancer (Nesslinger, et al., 2010), and Her2/neu vaccination for breast cancer (Disis, et al., 2004), and MAGE-A3 vaccination (Hardwick, et al., 2011; Corbiere, et al., 2011). Antigen spread has also been observed in response to immunotherapy treatment with a non-target-specific immunomodulator. For example, treatment of prostate cancer with the immunomodulator anti-CTLA4 (ipilimumab), which suppresses an immune system checkpoint can result in a broadened immune response (Kwek, et al., 2012). Accordingly, methods and compositions for measuring the extent of antigen spread in a patient undergoing immunotherapy such as ValloVax, treatment with a cancer cell or a mixture of antigens derived therefrom, or treatment with an immunomodulator are provided herein. Methods and compositions for predicting a positive therapeutic response to treatment are also provided. Such methods and compositions can include utilizing measurements of antigen spread. Such methods and compositions can also include measurements of the level of antibodies reactive to one or more predetermined biomarker antigens, such as the biomarker antigens provided herein. Such methods and compositions can further include measurements of the change in the level of antibodies reactive to one or more predetermined biomarker antigens, such as the biomarker antigens provided herein, in response to cancer treatment with an immunomodulator, treatment with a cancer cell or a mixture of antigens derived therefrom, or ValloVax. Moreover, methods and compositions for identifying new cancer antigens for development of additional endothelial targeting immune therapies are provided herein.

In another embodiment, a patient can be treated with cell specific active immunotherapy. For example, a patient can be treated with target cancer antigens that are a mixture of antigens derived from the patient's own tumor cells or allogenic tumor cells For example, one or more of the tumor cell lines, such as the prostate tumor LnCAP or PC-3 cell lines, can be killed, and a mixture of antigens (e.g., proteins) can be extracted therefrom. The mixture can be mixed with a pharmaceutical excipient and introduced into a patient. In some cases, the mixture is also combined with an adjuvant or an immunomodulator. In some cases, the treatment with syngenic or allogenic tumor cells can stimulate an immune response against (e.g., recognize, bind to, attack, opsonize, induce apoptosis or necrosis of, phagocytose, etc.) the patient's cancer cells. In some embodiments, an increase in one or more predetermined biomarker antigens as a result of treatment with a mixture of antigens derived from the patient's own tumor cells or allogenic tumor cells can predict a positive therapeutic outcome.

Means of utilizing the invention are applicable to existing cancer vaccines that utilize whole cells or lysates thereof in that existing cancer cell lines or vaccines can be modified to express endothelial antigens thus taking the shape of tumor-associated channels or tumor associated endothelium that is not derived from patient hematopoietic originating endothelial progenitor cells. Whole cancer cells may be allogeneic, syngeneic, or autologous to the treatment recipient. Typically they may be treated to make them proliferation incompetent by a technique which preserves preserve their immunogenicity and their metabolic activity. One typically used technique is irradiation. Such cells. Typically the same general type of tumor cell is used that the patient bears. For example, a patient suffering from melanoma will typically be administered proliferation incompetent melanoma cells. The cells may express and secrete a cytokine naturally or by transfection with a nucleic acid which directs such expression and secretion. One suitable cytokine is GM-CSF. For example, the tumor cell may express a transgene encoding GM-CSF as described in U.S. Pat. Nos. 5,637,483, 5,904,920, 6,277,368 and 6,350,445, as well as in US Patent Publication No. 20100150946, each of which is expressly incorporated by reference. One example of a GM-CSF-expressing, genetically modified cancer cell for the treatment of pancreatic cancer is described in U.S. Pat. Nos. 6,033,674 and 5,985,290, both of which are expressly incorporated by reference herein. Other cytokines can be used. Suitable cytokines which may be used include cytokines which stimulate dendritic cell induction, recruitment, and/or maturation. Such cytokines include, but are not limited to, one or more of GM-CSF, CD40 ligand, IL-12, CCL3, CCL20, and CCL21. Granulocyte-macrophage colony stimulating factor (GM-CSF) polypeptide is a cytokine or fragment having immunomodulatory activity and having at least about 85% amino acid sequence identity to GenBank Accession No. AAA52122.1.

According to one alternative embodiment, checkpoint inhibitors such as antibodies to CTLA4, LAG3, PD1, or PD1 ligand are delivered by inactivated bystander cells which express and secrete one or more cytokines. The bystander cell may be a monocyte, a fibroblast, or a proliferating endothelial progenitor cell.

Alternatively bystander cells may be mesenchymal stem cells transfected with cytokines or Type 1 mesenchymal stem cells. The bystander cells may provide all of the antibodies, or single chain antibodies which act as checkpoint inhibitors. In addition, immunomodulatory cytokine-expressing bystander cell lines can be used to overcome immune suppression as a means of augmenting ValloVax activity, such cell lines are described in U.S. Pat. Nos. 6,464,973, and 8,012,469, Dessureault et al., Ann. Surg. Oncol. 14: 869-84, 2007, and Eager and Nemunaitis, Mol. Ther. 12:18-27, 2005, each of which is expressly incorporated by reference.

In yet another embodiment, a patient can be treated with an immunomodulator, such as one or more immunomodulators described herein. In some cases, treatment with an immunomodulator that activates the immune system or inhibits a suppressor (e.g., a checkpoint) of the immune system can result in increased immune surveillance or activity (e.g., recognition, binding to, opsonization of, induction of apoptosis or necrosis, phagocytosis, etc.) against a patient's cancer cells. In some cases, an increase in one or more predetermined biomarker antigens as a result of treatment with an immunomodulator can predict a positive therapeutic outcome. Subsequent to immune derepression ValloVax or other tumor endothelium targeting vaccines are administered.

In yet another embodiment, self-antigen reactive antibody levels of a patient or population of patients suffering from cancer can be measured and correlated with overall survival. Levels of antibodies that are reactive to a particular biomarker antigen or group of biomarker antigens that correlate with improved overall survival can then identify that antigen or group of antigens as target antigens for cancer immunotherapy. In some cases, an increase in the levels of antibodies reactive to one or more biomarker antigens that correlates with improved overall survival can identify those one or more biomarker antigens as target antigens for cancer immunotherapy. In some cases, the increase in the levels of antibodies reactive to one or more biomarker antigens is an increase from pre-treatment levels to post-treatment levels. In some cases, the biomarker antigens include, or are, any one or more of PSA, KLK2, KRAS, ERAS, LGALS8, LGALS3, PAP, or PAP-GM-CSF, individually or in any combination, such as any of the foregoing combinations described herein. In some cases, non-target predetermined biomarker antigens are measured and compared to determine the presence, absence, or degree of increase in reactive antibodies. In some cases, the non-target predetermined biomarker antigens include, or are, any one or more of KLK2, KRAS, ERAS, LGALS8, LGALS3, or PSA, individually or in any combination, such as any of the foregoing combinations described herein.

The invention protocol uses an immunomodulatory and conditioning regimen that will enhance both the induction and effector phases of the immune response, as well as, radiation induced upregulation of tumor neovascular adhesion molecules, combined with a cancer endothelium vaccine for the treatment of tumors.

It is known the efficacy of the induction phase can be improved by blocking the negative regulators of the activation of effector T cells (Korman, et al., (2005)). Cytotoxic T cell associated antigen-4 (CTLA-4) is expressed on activated T cells as a regulatory brake that halts T cell activation. Blocking the activity of CTLA-4 allows greater expansion of all T cell populations, presumably including those with anti-tumor activity.

Before or during the endothelium vaccination protocol, the subjects are subjected to radiation directed at the tumor or, in some cases, to whole body irradiation. The effect of this radiation treatment is to induce remodeling of the vasculature so that extravasation of effector T cells into the tumor is enhanced. If the tumor to be treated is not a solid tumor or a tumor with defined lesions, this aspect of the protocol is optional and generally unnecessary. The effect of radiation is to ease the entry of the effector T cells elicited by the vaccine into solid tumors, so that the radiation can be administered immediately before or during the vaccination protocol. The level of radiation dosage will depend on whether the tumor is targeted directly or whole body radiation is employed and on the level of remodeling that needs to be effected. The radiation schedule can be integrated with the schedule for administration of the vaccine and with the schedule for the administration of anti-CTLA-4 antibody that modulates the effect of Tregs. Each of the radiation treatments may be scheduled at a time selected to correspond to a particular administration of the vaccine and/or the Tregs modulator.

In aspects of the invention, the immunostimulatory oligonucleotides can encompass various chemical modifications and substitutions, in comparison to natural RNA and DNA, involving a phosphodiester internucleoside bridge, a .beta.-D-ribose unit and/or a natural nucleoside base (adenine, guanine, cytosine, thymine, uracil). Examples of chemical modifications are known to the skilled person and are described, for example in Uhlmann E. et al. (1990), Chem. Rev. 90:543; “Protocols for Oligonucleotides and Analogs” Synthesis and Properties & Synthesis and Analytical Techniques, S. Agrawal, Ed., Humana Press, Totowa, USA 1993; Crooke, S. T. et al. (1996) Annu. Rev. Pharmacol. Toxicol. 36:107-129; and Hunziker J. et al., (1995), Mod. Synth. Methods 7:331-417.

In one embodiment, the radiation is conducted immediately preceding (e.g., about 12 hours-36 hours) the administration of the Tregs modulator. The parameters of the irradiation are designed to have the effect of enhancing an immune response, rather than directly treating the tumor itself.

The vaccination protocol itself employs any vaccine directed to eliciting an immune response to a tumor associated antigen. As noted above, the vaccine may be in the form of protein, nucleic acid, or autologous or allogeneic cells and may be univalent or multivalent. Techniques for administering antigens designed to elicit, in particular, a cellular response are well known. Typically, the administration of such vaccines is parenteral, typically intravenous. Depending on the vaccine chosen, the administration may be over a period of minutes, hours or days.

During the vaccination protocol, the function of Tregs is modulated according to the method of the invention, while the effectiveness of the effector T cells generated by the vaccine is unaffected. Thus, agents need to be chosen that are specific for Tregs as opposed to targeting effector T cells in general. One such agent that is particularly favored is anti-CTLA-4. Monoclonal antibodies that have this function are available, including tremelimumab which is an IgG1 human mAb and an alternative IgG1 human monoclonal mAb, ipilimumab. However, other agents that specifically target the function of Tregs while not substantially inhibiting antitumor T cells may be substituted. For example, any binding agent for CTLA-4 may be used, such as aptamers or other specific binding partners.

It should be noted that although mAb's are commercially available and convenient, mAb's per se would not be required. Clearly fragments of such antibodies, recombinantly produced forms, such as single-chain antibodies, antibody mimics, such as aptamers, and the various art-known modifications of traditional antibodies can be included. Thus, the CTLA-4 binding agent employed may include any of these functionalities. Anti-CTLA-4 antibodies thus include mimics, fragments, and various recombinantly produced or modified forms of native antibodies. If the vaccine includes allogeneic or autologous cells, these cells may be modified to produce the CTLA-4 binding agents as well.

### Example 1: Synergy of CTLA-4 Blockade and ValloVax

Full term human placentas were collected from delivery room under informed consent. Fetal membranes were manually peeled back and the villous tissue is isolated from the placental structure. Villous tissue was subsequently washed with cold saline to remove blood and scissors used to mechanically digest the tissue. Lots of 25 grams of minced tissue were incubated with approximately 50 ml of HBSS with 25 mM of HEPES and 0.28% collagenase, 0.25% dispase, and 0.01% DNAse at 37 Celsius. The mixture of minced placental villus tissue and digesting solution was incubated under stirring conditions for three incubation periods of 20 minutes each. Ten minutes after the first incubation period and immediately after the second and third incubation periods, the DNAse was added to make up a total concentration of DNase, by volume, of 0.01%. In the first and second incubations, the incubation flask is set at an angle, and the tissue fragments allowed to settle for approximately 1 minute, with 35 ml of the supernatant cell suspension being collected and replaced by 38 ml (after the first digestion) or 28 ml (after the second digestion) of fresh digestion solution. After the third digestion the whole supernatant was collected. The supernatant collected from all three incubations was then pooled and is poured through approximately four layers of sterile gauze and through one layer of 70 micrometer polyester mesh. The filtered solution was then centrifuged for 1000 g for 10 minutes through diluted new born calf serum, said new born calf serum diluted at a ratio of 1 volume saline to 7 volumes of new born calf serum. The pooled pellet was then resuspended in 35 ml of warm DMEM with 25 mM HEPES containing 5 mg DNase I. The suspension was subsequently mixed with 10 ml of 90% Percoll to give a final density of 1.027 g/ml and centrifuged at 550 g for 10 minutes with the centrifuge brake off. The pellet was then washed in HBSS and cells incubated for 48 hours in complete DMEM media containing 100 IU of IFN-gamma per ml. Subsequent to incubation cells were mitotically inactivated by irradiation at 10 Gy and used for administration.

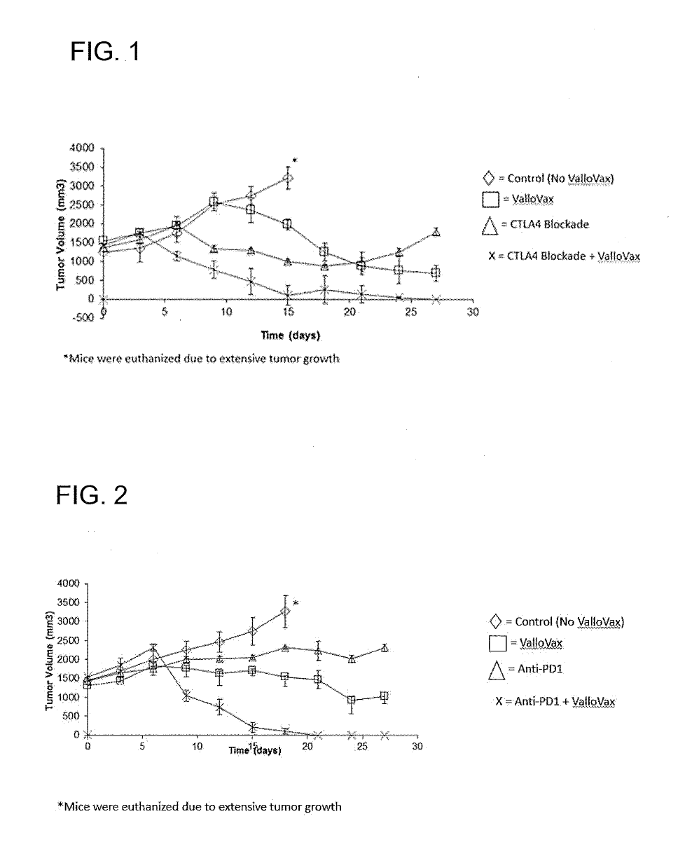

For induction of tumor growth, 5×105 B16, LLC cells purchased from American Type Culture Collection (Manassas, Va.) cells were injected subcutaneously into the hind limb flank. Tumors were allowed to grow for 12 days, after which weekly vaccinations of 5×105 ValloVax cells were administered subcutaneously on the contralateral side to which tumors were administered. Anti CTLA-4 antibody (FIG. 1) or anti PD-1 antibody (FIG. 2) were given every second day.

Tumor growth was assessed every 3 days by two measurements of perpendicular diameters by a caliper, and animals were sacrificed when tumors reached a size of 1 cm in any direction. Tumor volume was calculated by the following formula: (the shortest diameter2×the longest diameter)/2.

Synergistic inhibition of tumor growth was observed between checkpoint blockade and ValloVax treatment.

1. A method of inducing tumor regression comprising the steps of:
a) administering an immunogen capable of stimulating an immune response with selectivity to tumor associated endothelium;
b) administering a checkpoint inhibitor.

2. The method of claim 1, wherein said immunogen capable of stimulating immunity towards tumor associated endothelium is selected from a group of cellular immunogens comprising of: a) endothelial progenitor cells; b) placental endothelial cells; c) tumor differentiated vascular channel cells; d) progenitor cells differentiated into endothelial cells.

3. The method of claim 1, wherein said immunogen capable of stimulating immunity towards tumor associated endothelium is selected from a group of protein immunogens comprising of:
a) TEM-1;
b) ROBO-4;
c) ROBO 1-18;
d) VEGFR2;
e) CD109;
f) survivin; and
g) CD93.

4. The method of claim 2, wherein said cellular immunogens are pretreated with an agent capable of augmenting immunogenicity of said cellular immunogens.

5. The method of claim 4, wherein said agents capable of augmenting immunogenicity increase expression of an HLA or HLA-like molecule.

6. The method claim 4, wherein said agents capable of augmenting immunogenicity increase expression of costimulatory molecules.

7. The method of claim 6, wherein said costimulatory molecules are selected from a group comprising of:
a) CD40;
b) CD 80;
c) CD86;
d) OX40;
e) ICOS; and
f) 4-1 BB.

8. The method of claim 4 wherein said agents capable of augmenting immunogenicity are selected from a group comprising of:
a) IL-1;
b) IL-2;
c) TNF-alpha;
d) IFN-gamma;
e) IL-33; and
f) IL-27.

9. The method of claim 4, wherein augmentation of immunogenicity is achieved by exposure of said cells to sublethal hyperthermia.

10. The method of claim 9, wherein said sublethal hyperthermia is sufficient to augment expression of heat shockproteins in said cell.

11. The method of claim 10, wherein said heat shock proteins are selected from a group comprising of:
a) gp96;
b) hsp 35;
c) hsp 70; and
d) hsp 95.

12. The method of claim 4, wherein said immunogenicity is augmented by coculture of said immunogen cells with allogeneic T cells.

13. The method of claim 12, wherein said coculture is sufficient to induce production of more than 50 picograms per ml in a culture of 100,000 immunogen cells with 100,000 allogeneic T cells in a volume of 200 microliters.

14. The method of claim 13, wherein said coculture is performed for 48 hours.

15. The method of claim 14, wherein said immunogen cells are selectively purified after said culture with T cells and subsequently used for treatment.

16. The method of claim 15, wherein isolation of said immunogen cells from said T cells is performed by an isolation means selected from a group comprising of:
a) magnetic activated cell sorting;
b) flow cytometry sorting; and
c) cell panning.

17. The method of claim 2, wherein said endothelial progenitor cells are purified from a source selected from a group comprising of:
a) cord blood endothelial progenitor cells;
b) circulating endothelial progenitor cells;
c) bone marrow endothelial progenitor cells; and
d) placental matrix endothelial progenitor cells.

18. The method of claim 17, wherein said endothelial progenitor cells are capable of forming endothelial colonies when cultured in a matrigel substrate.

19. The method of claim 17, wherein said endothelial progenitor cells are capable of forming endothelial colonies when cultured in a methylcellulose substrate.

20. The method of claim 17, wherein said endothelial progenitor cells are capable of forming blood vessel-like tubes when implanted in an immune deficient mouse.

21.-115. (canceled)
